(19) United States
(12) Patent Application Publication
(10) Pub. No.: US 2020/0390979 A1
(43) Pub. Date: Dec. 17, 2020

(54) DEVICE FOR SECURING PROTECTIVE COVER OF SYRINGE

(71) Applicant: Man-Pun Yau, Pingtung (TW)

(72) Inventor: Man-Pun Yau, Pingtung (TW)

(21) Appl. No.: 16/438,419

(22) Filed: Jun. 11, 2019

(51) Int. Cl. A61M 5/32

(52) U.S. Cl. CPC A61M 5/3204; A61M 2209/04; A61M 2205/586

(57) ABSTRACT

A device for securing a protective cover of a syringe includes a body which includes a fixing portion and a grip portion. The fixing portion has at least one clamp formed on the top thereof. The protective cover of the syringe is secured by the at least one clamp so that a safe distance is obtained between the protective cover and the user's finger. The user can easily and conveniently insert the needle into the protective cover.

### BACKGROUND OF THE INVENTION

### 1. Fields of the invention

The present invention relates to a protective cover, and more particularly, to a device for securing the protective cover of a syringe so as to keep a distance between the user's fingers and the protective cover when inserting the needle into the protective cover.

### 2. Descriptions of Related Art

The conventional syringe includes a barrel with a needle adapter on the first end thereof, and a needle is formed to the needle adapter. A plunger is movably inserted into the barrel from the second end of the barrel so as to inject medicine in the barrel into the patient body via the needle. Generally, when the injection is completed, the users will insert the needle of the syringe into the protective cover. It is a risk for the users if the needle accidentally touches the users' skin during the insertion of the needle back into the protective cover.

The present invention intends to provide a device for securing the protective cover of a syringe, so that the users can conveniently aim the needle to the protective cover and successfully insert the needle into the protective cover.

### SUMMARY OF THE INVENTION

The present invention relates to a device for securing a protective cover of a syringe, and comprises a body having a fixing portion and a grip portion. The fixing portion has at least one clamp formed on the top thereof.

Preferably, the body is a plate.

Preferably, the at least one clamp includes two clamping plates located at a distance from each other. At least one protrusion protrudes from the inside of each of the two clamping plates so as to secure the protective cover.

Preferably, the fixing portion includes first clip formed to the underside thereof so as to clip the board to another object.

Preferably, the grip portion includes a protective plate extending upward from one side thereof.

Preferably, the grip portion includes a connection member connected thereto, and the connection member is connected with a second clip. The second clip clips the device to the user's pocket.

The primary object of the present invention is to provide a device for securing a protective cover of a syringe, wherein the protective cover is secured by the at least one clamp of the device such that a safe distance is obtained between the protective cover and the user's finger. The user is easily and conveniently inserts the needle into the protective cover. The device is compact and light in weight. The device is easily used and manufactured at low cost. The device is easily carried by every user.

The present invention will become more apparent from the following description when taken in connection with the accompanying drawings which show, for purposes of illustration only, a preferred embodiment in accordance with the present invention.

### BRIEF DESCRIPTION OF THE DRAWINGS

FIG. 1 is a perspective view to show the device of the present invention;

FIG. 2 is another perspective view to show the device of the present invention;

FIG. 3 shows that the protective cover is clamped by the clamps of the device, and the needle is inserted in the protective cover;

FIG. 4 shows that the protective cover is clamped by the clamps of the device, and the needle is to be inserted in the protective cover;

FIG. 5 shows that the user holds the grip portion of the device by one hand, and uses the other hand to insert the needle into the protective cover;

FIG. 6 shows that the user holds the grip portion of the device by one hand, and the needle is inserted into the protective cover by the other hand;

FIG. 7 shows that the user holds and lifts the barrel of the syringe to remove the syringe with the protective cover from the clamps of the device of the present invention, and

FIG. 8 shows that the device of the present invention is clipped to a board.

### DETAILED DESCRIPTION OF THE PREFERRED EMBODIMENT

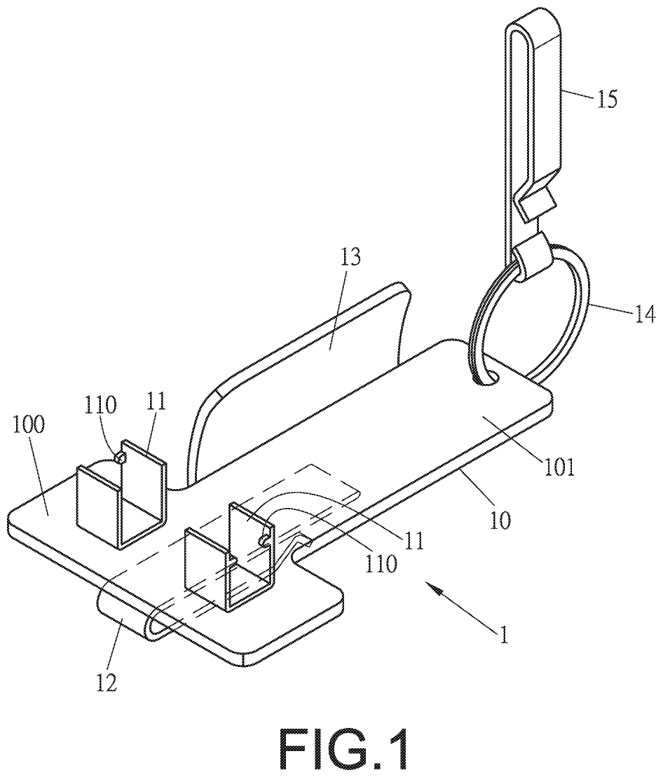

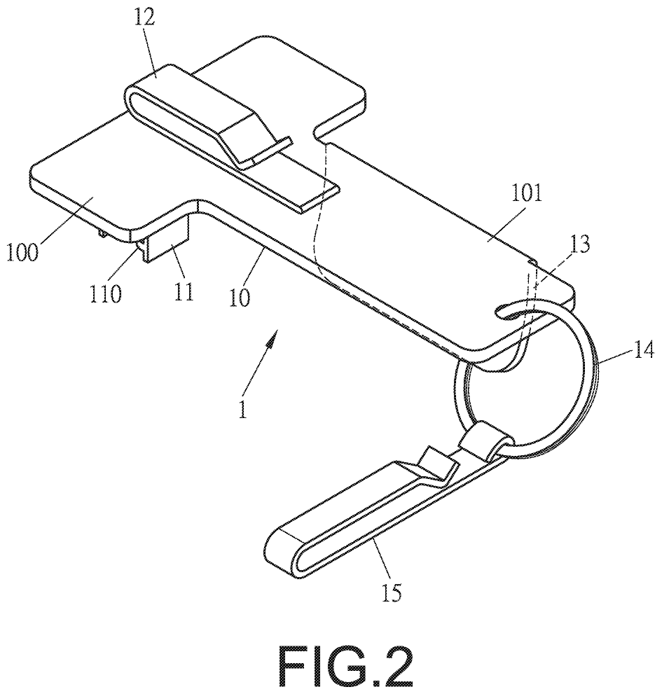

Referring to FIGS. 1 and 2, the device for securing a protective cover of a syringe of the present invention comprises a body 10 having a fixing portion 100 and a grip portion 101. The fixing portion 100 has two clamps 11 formed on the top thereof. Each clamp 11 includes two clamping plates located at a distance from each other. A protrusion 110 protrudes from the inside of each of the two clamping plates, A first clip 12 is formed to the underside of the fixing portion 100. The grip portion 101 further includes a protective plate 13 extending upward from one side thereof. A connection member 14 is connected to the grip portion 101. The connection member 14 is connected with a second clip 15. Specifically, the fixing portion 100 and the grip portion 101 form the body 10 as a T-shaped plate.

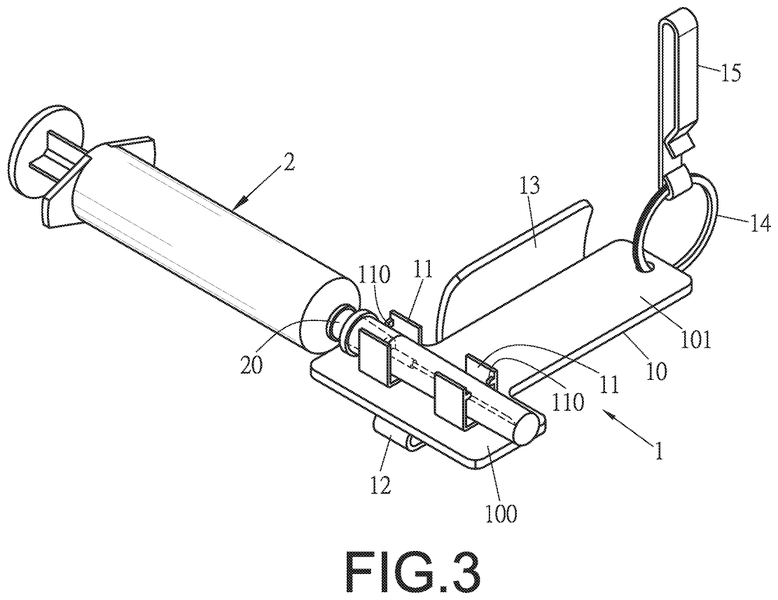

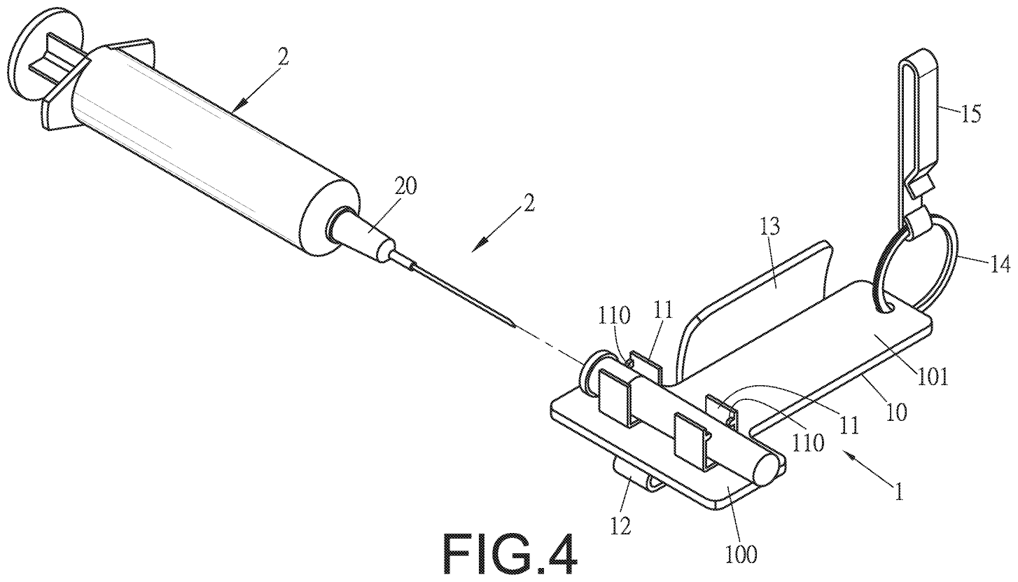

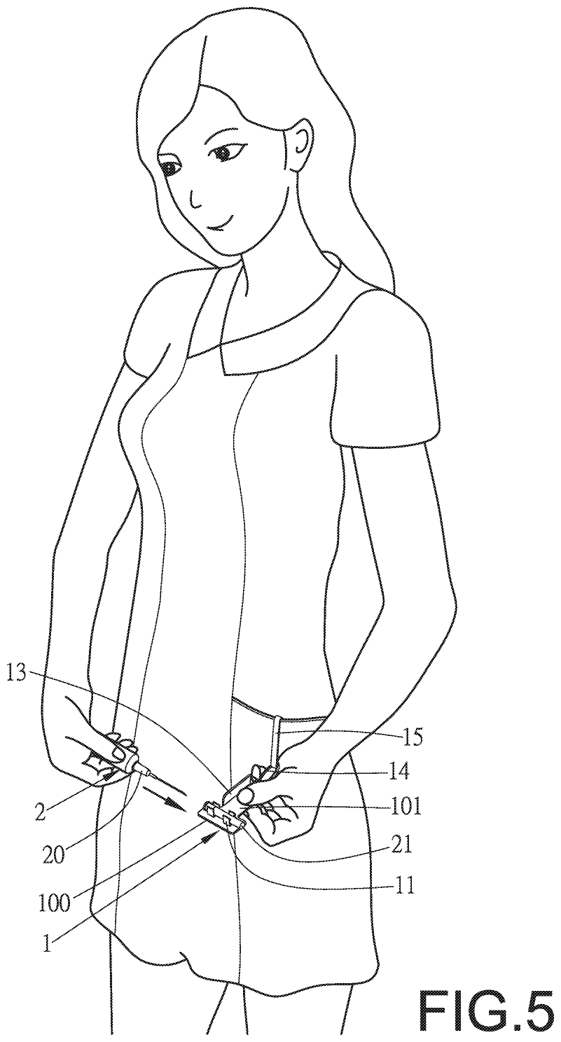

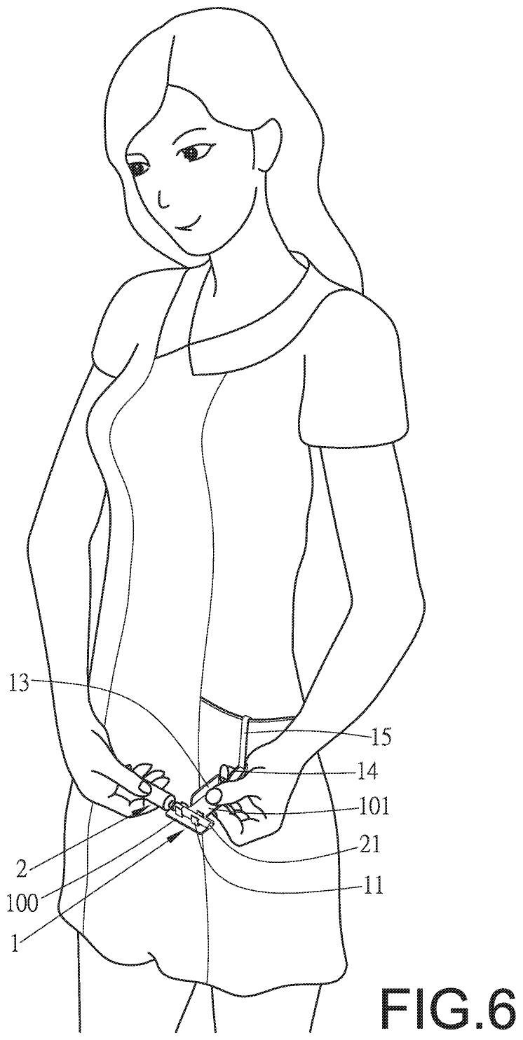

As shown in FIGS. 3 to 8, the second clip 15 clips the body 10 to the user's pocket as shown in FIGS. 5 to 7 so that the device 1 is easily carried. Before using the syringe 2, the user holds the barrel of the syringe 2 and pushes the protective cover 21 into the two clamps 11 on the fixing portion 100 as shown in FIG. 3, wherein the needle 20 is inserted in the protective cover 21. The protective cover 21 is well clamped by the protrusions 110 of the two clamps 11. As shown in FIG. 4, the user pulls the barrel away from the protective cover 21 so that the needle 20 is removed from the protective cover 21. The user then pushes the plunger to inject medicine into the patient's body. After injection, the user holds the grip portion 101 of the body 10 of the device 1 by one hand, and the other hand holds the syringe 2 and aims the needle 20 to the protective cover 21. The user's fingers are located away from the protective cover 21 and the needle 20, and the needle 20 is easily inserted into the protective cover 21 as shown in FIGS. 5 and 6. The protective plate 13 on one side of the grip portion 101 prevents the user's fingers from being hurt by the needle 20 when the needle 20 is inserted into the protective cover 21.

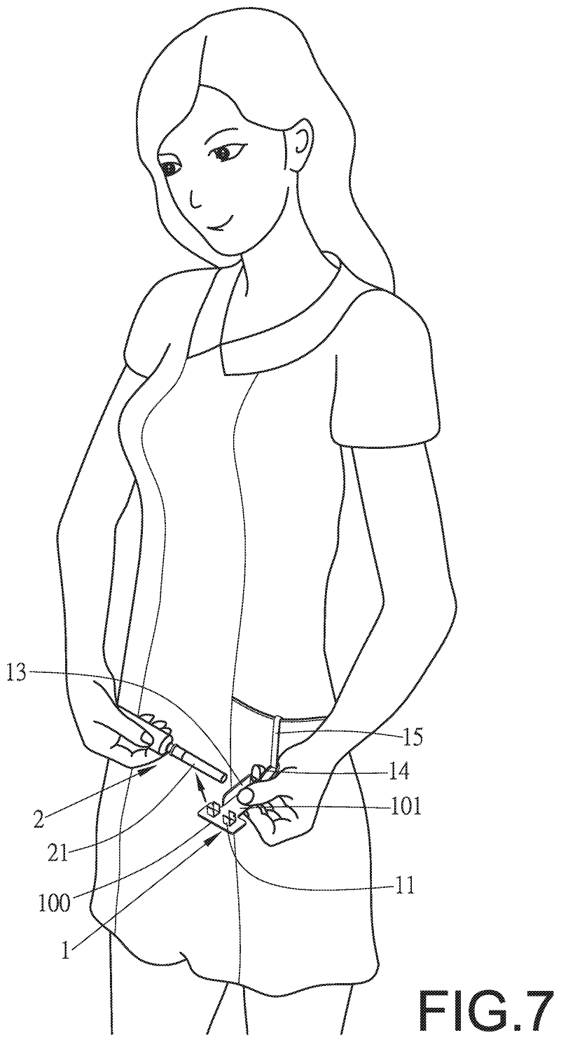

After the needle 20 is inserted into the protective cover 21, the user lifts the syringe 2 away from the body 10 so that the protective cover 21 slightly pushes the two clamping plates of each clamp 11 apart from each other to separate the syringe 2 from the device 1 as shown in FIG. 7.

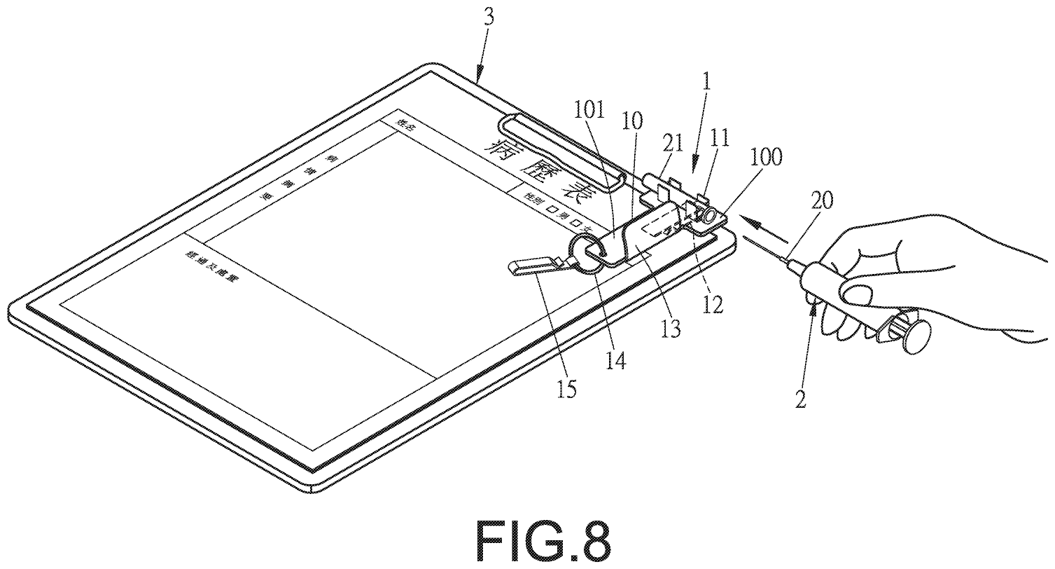

When the user operates the syringe 2 by one hands, and the other hand needs to press the user's body, the device 1 can be clipped to an object such as a board 3, by the first clip 12 as shown in FIG. 8. Therefore, the user can use one hand to insert the needle 20 back into the protective cover 21.

It is noted that the device 1 is compact and light in weight, and the device 1 is easily used and manufactured at low cost. The device 1 is easily carried by every user.

While we have shown and described the embodiment in accordance with the present invention, it should be clear to those skilled in the art that further embodiments may be made without departing from the scope of the present invention.

What is claimed is:

1. A device for securing a protective cover of a syringe, comprising:
a body having a fixing portion and a grip portion, the fixing portion having at least one clamp formed on a top thereof.

2. The device for securing a protective cover of a syringe as claimed in claim 1, wherein the body is a plate.

3. The device for securing a protective cover of a syringe as claimed in claim 1, wherein the at least one clamp includes two clamping plates located at a distance from each other, at least one protrusion protrudes from an inside of each of the two clamping plates.

4. The device for securing a protective cover of a syringe as claimed in claim 1, wherein the fixing portion includes a first clip formed to an underside thereof.

5. The device for securing a protective cover of a syringe as claimed in claim 1, wherein the grip portion includes a protective plate extending upward from one side thereof.

6. The device for securing a protective cover of a syringe as claimed in claim 1, wherein the grip portion includes a connection member connected thereto, the connection member is connected with a second clip.
